(19) United States
(12) Patent Application Publication
(10) Pub. No.: US 2021/0113801 A1
(43) Pub. Date: Apr. 22, 2021

(54) SYSTEMS AND METHODS FOR PROVIDING CONCENTRATED OXYGEN TO A USER

(71) Applicant: ROAM TECHNOLOGIES PTY LTD, Summer Hill NSW (AU)

(72) Inventors: Shan-Shan WANG, Strathfield NSW (AU); Nicholas James BARONI, Mosman NSW (AU); Eugene LAI, Labor Park NSW (AU); Dylan LAW, Denistone West NSW (AU); Andrew Wagih KHALIL, Kangaroo Point (AU)

(73) Assignee: ROAM TECHNOLOGIES PTY LTD, Summer Hill NSW (AU)

(21) Appl. No.: 17/072,508

(22) Filed: Oct. 16, 2020

Related U.S. Application Data
(63) Continuation of application No. PCT/IB2019/000417, filed on Apr. 12, 2019.
(60) Provisional application No. 62/660,421, filed on Apr. 20, 2018.

(51) Int. Cl. A61M 16/10; A61M 16/00; A61M 16/20

(52) U.S. Cl. CPC A61M 16/101; A61M 2230/432; A61M 16/0063; A61M 16/1015; A61M 16/201; A61M 16/105; A61M 16/0051; A61M 16/0003; A61M 2205/3334; A61M 2205/02; A61M 2205/502; A61M 2205/3553; A61M 2230/205; A61M 2230/10; A61M 2230/04; A61M 16/0093

(57) ABSTRACT

The embodiments of the present disclosure provide a portable oxygen concentrator. The portable oxygen concentrator may comprise an input configured to receive airflow, an input filter, a compressor configured to compress airflow, a first column comprising a first adsorbent bed, and a second column adjacent to the first column and comprising a second adsorbent bed. The portable oxygen concentrator may further comprise a first output configured to release oxygen to a user, and a second output configured to release waste gas. The first and second adsorbent beds may comprise a plurality of zeolites.

### CROSS-REFERENCE TO RELATED PATENT APPLICATIONS

This application claims priority under 35 U.S.C. § 119 to U.S. Provisional Patent Application No. 62/660,421 , filed Apr. 20, 2018, the disclosure of which is expressly incorporated herein by reference in its entirety.

### BACKGROUND

Current oxygen delivery systems available on the market are heavy, costly and require ongoing maintenance, and contain enticing dials and switches that allow the user to alter the flow rate settings. Further, current technologies are limited in maximum dose capabilities and oxygen purity levels. Moreover, current portable oxygen concentrators (POCs) in market provide a manual control of oxygen output. As such patients select a preset output liters per minute (LPM) (typically 1 to 5 LPM) when the doctor prescribes. Usually, the patient will be prescribed to set a lower LPM setting when idle or resting, and a higher LPM when anticipating to perform a strenuous activity.

Medical devices to date have also been narrowly focused on providing a singular “static” solution focused on linear treatment of diseases. Unfortunately, diseases and their required treatment do not always stay within one discipline or organ. Similarly, when physicians do not communicate, tests are duplicated, time is wasted, and money is lost.

In addition, while current POCs utilize adsorbent beds, such as zeolite beds, the amount of utilization is around 25%. As such, as the mass transfer zone (MTZ) reaches the end of the zeolite beds, the performance of zeolite adsorption becomes ineffective. Therefore, there is a need for improved POCs that reduce the MTZ, allow the MTZ to stay in the zeolite beds longer, and thereby allow more zeolites to undergo adsorption.

The embodiments of the present disclosure provide a portable oxygen concentrator (POC) that can be built specifically to accomplish the objectives of both patients' needs and doctors' wants with corrected dose volume and oxygen purity, thereby meeting patient's needs at any activity levels.

The increase of people living longer and the demand for higher quality of life are driving growth in the healthcare sector. Conditions such as hypoxemia (insufficient oxygen in the blood), particularly chronic obstructive pulmonary disease (COPD), asthma, pneumonia, heart failure, major trauma and obstetric emergencies as such require an effective oxygen delivery system that integrates with the user's lifestyle.

This trend is underpinned by the following megatrends:
Increase of people living longer
Demand for higher quality of life and the flexibility of the medical devices to be “smart” and adapt to the users' needs
The need for medical devices to have analytics to assist clinicians and patients to monitor their condition, whilst undertaking a variety of normal daily activities.
Rise in air pollution resulting to increase in oxygenated healthcare
Desire for simplified lightweight technologies
Increased focus on performance and efficiency

### SUMMARY

According to the exemplary embodiments of the present disclosure, a portable oxygen concentrator is provided. The portable oxygen concentrator may comprise an input configured to receive air flow, an input filter, a compressor configured to compress air flow, a first column comprising a first adsorbent bed, a second column adjacent to the first column, a first output configured to release oxygen to a user, and a second output configured to release waste gas. The second column may comprise a second adsorbent bed. The first and second adsorbent beds may each comprise a plurality of zeolites.

In another embodiment, the portable oxygen concentrator may further comprise a top manifold at a distal end of the first and second columns, and a bottom manifold at a proximal end of the first and second columns. The top and bottom manifolds may comprise the first and second outputs and an internal network of tubes configured to allow air flow. In other aspects, the top and bottom manifolds may further comprise a plurality of solenoid valves configured to control the flow of air. In yet another embodiment, the top and bottom manifolds may further comprise a plurality of holes configured to control a flow rate of air. The top and bottom manifolds may be made with various materials, including a metal alloy, or polymeric material such as plastic or resin. The top and bottom manifolds may be made via injection molding, computer numerical control (CNC), or 3D printing (additive manufacturing).

According to another embodiment, the input of the portable oxygen concentrator may have a first diameter that is bigger than a second diameter of the second output. In other aspects, the top and bottom manifolds may comprise a plurality of check valves that are configured to seal the plurality of holes.

The first and second columns of the portable oxygen concentrator may comprise at least one of aluminum or thermoplastic. In other aspects, the first and second columns may vary in shape. For example, the first and second columns may be cylindrical, rectangular, or triangular in shape. In some embodiments, the first and second columns may be 3D printed. In another embodiment, a proximal end of the first and second columns may be coupled to the first output, and a distal end of the first and second columns may be coupled to the compressor. The first and second columns may each comprise an O-ring coupled to at least one of the proximal end or the distal end.

In other embodiments, the portable oxygen concentrator may further comprise a cap or a lid at the proximal end and the distal end of the first and second columns. The cap or the lid may comprise a tapered air flow path. In another embodiment, the portable oxygen concentrator may comprise at least one sintered glass filter disc at the proximal end and the distal end of the first and second columns. The sintered glass filter disc may be configured to filter the plurality of zeolites from the compressed air. In other embodiments, the portable oxygen concentrator may further comprise a wave spring located in between the cap and the at least one sintered glass filter disc. The wave spring may be configured to compress the plurality of zeolites in the first and second columns. Alternatively, the portable oxygen concentrator may comprise a dense foam material located in the cap. The dense foam material may be configured to compress the plurality of zeolites in the first and second columns. In yet another embodiment, the portable oxygen concentrator may comprise a rubber durometer located in the cap. The rubber durometer may be configured to compress the plurality of zeolites in the first and second columns.

In another embodiment, the portable oxygen concentrator may further comprise at least one sensor and a processor. The sensor may be configured to detect at least one physiological parameter of the user. The processor may be configured to adjust an amount of oxygen released to the user based on the detected at least one physiological parameter. In some embodiments, the sensor may comprise at least one of a pulse oximeter, differential pressure sensor, ECG, EEG, gyroscope, accelerometer, or any combination thereof. The physiological parameter of the user detected may comprise at least one of volume of air breath, CO₂concentration in air exhaled, SpO₂ concentration, heart rate, pulse rate, average breaths per minute, inhale pressure, exhale pressure, sound of breath, or any combination thereof. In some embodiments, the portable oxygen concentrator may comprise a printed circuit board (PCB) coupled to the compressor, and the at least one sensor may be coupled to the PCB. In other embodiments, the processor may be configured to generate an alarm when the detected at least one physiological parameter is above or below a predetermined threshold.

According to the embodiments of the present disclosure, the plurality of zeolites may comprise at least one of LiLSX zeolites, LiAgX zeolites, AgX zeolites, NaX zeolites, or CaA zeolites. For example, the plurality of zeolites may comprise at least an activated alumina composition and an LiLSX composition. In some embodiments, the activated alumina composition may comprise at least one of Ai₂θ3, Na₂O , Fe₂O3, TiO₂, or SiO₂ In other embodiments, the LiLSX composition may comprise at least one of zeolite, cuboidal, crystalline, synthetic, non-fibrous, mineral binder, or Quartz (SiO₂).

According to another embodiment, the first column of the portable oxygen concentrator may be configured to provide oxygen to the first output when the second column is configured to release waste gas to the second output. The first column may be further configured to release waste gas to the second output when the second column is configured to provide oxygen to the first output. In other embodiments, the first and second columns may have a diameter to length ratio of about 1:6. In other aspects, the first and second columns may each comprise between about 20 and about 80 grams of zeolites. In some embodiments, the pressure inside the first and second columns may be maintained between about 1 bar pressure and about 5 bar pressure. For example, the pressure inside the first and second columns may be maintained between about 1.25 bar pressure and about 2 bar pressure. In some embodiments, the first and second columns may be configured to allow air to flow radially to thereby channel the air through the first and second columns and increase contact with the plurality of zeolites in the first and second adsorbent beds.

The portable oxygen concentrator may further comprise a user interface configured to receive user input. The processor may be configured to adjust the amount of oxygen released to the user based on the user input received. In other embodiments, the portable oxygen concentrator may comprise a wireless receiver configured to receive data from a remote device. The processor may be configured to adjust the amount of oxygen released to the user based on the received data. The remote device may comprise at least one of a computer, a smartphone, a wearable device, or any combination thereof. In some embodiments, the portable oxygen concentrator may further comprise a removable battery coupled to the first and second columns.

According to another embodiment of the present disclosure, a method of providing concentrated oxygen to a user is provided. The method may comprise directing and compressing air into a first column of an oxygen concentrator. The first column may comprise a first adsorbent bed. The method may further comprise absorbing nitrogen and oxygen molecules from the air in the first adsorbent bed, and directing and compressing the air into a second column of an oxygen concentrator adjacent to the first column. The second column may comprise a second adsorbent bed. The method may further comprise absorbing nitrogen and oxygen molecules from the air in the second adsorbent bed and depressurizing the first column. Depressurizing the first column may allow the argon and nitrogen molecules in the first column to be purged out of the oxygen concentrator and released to the atmosphere. The method may further comprise directing and compressing the air into the first column and depressurizing the second column. Depressurizing the second column may allow the argon and nitrogen molecules in the second column to be purged out of the oxygen concentrator and released to the atmosphere. In some embodiments, depressurizing the first column, and directing and compressing the air into the second column may be performed concurrently.

According to another embodiment of the present disclosure, a zeolite composition for providing concentrated oxygen to a user is provided. The zeolite composition may comprise an activated alumina composition and an LiLSX composition. The weight ratio of the activated alumina composition to the LiLSX composition may be in a range of about 0.2 to about 0.5. In some aspects, the LiLSX composition may comprise a plurality of first pellets. The first pellets may each have a size of about 0.4 mm and a mesh size of about 30×60. In other aspects, the activated alumina composition may comprise a plurality of second pellets. The second pellets may each have a size of about 0.5 mm and a mesh size of about 28×48.

### BRIEF DESCRIPTION OF THE DRAWINGS

FIG. 1 illustrates various components of an exemplary oxygen delivery system, according to the embodiments of the present disclosure.

FIGS. 2A-2D illustrate the steps of pressure swing adsorption (PSA) in a two-column system, according to the embodiments of the present disclosure.

FIG. 3 is a partial perspective view of an exemplary device, according to the embodiments of the present disclosure.

FIG. 4 is a partial perspective view of a top manifold design of an exemplary device, according to the embodiments of the present disclosure.

FIG. 5 is a partial perspective view of a bottom manifold design of an exemplary device, according to the embodiments of the present disclosure.

FIGS. 6A-6D illustrate the steps of pressure swing adsorption (PSA) in a two-column system, according to the embodiments of the present disclosure.

FIGS. 7A-7E illustrate the steps of pressure swing adsorption (PSA) in a two-column system, according to the embodiments of the present disclosure.

FIGS. 8A-8D illustrate the steps of pressure swing adsorption (PSA) in a two-column system, according to the embodiments of the present disclosure.

FIGS. 9A-9E illustrate the steps of pressure swing adsorption (PSA) in a two-column system, according to the embodiments of the present disclosure.

FIG. 10 is a partial perspective view of a two-column system of an exemplary device, according to the embodiments of the present disclosure.

FIG. 11A graphically illustrates a pulse flow of oxygen delivered by current portable oxygen concentrator (POC) devices.

FIG. 11B graphically illustrates a continuous flow of oxygen delivered by an exemplary device, according to the embodiments of the present disclosure.

FIG. 12 illustrates a vessel/column of an exemplary device, according to the embodiments of the present disclosure.

FIG. 13 illustrates a cross-sectional view of an exemplary device, according to the embodiments of the present disclosure.

FIG. 14 illustrates a wave spring of an exemplary device, according to the embodiments of the present disclosure.

FIG. 15 illustrates cross-sectional view of a column of an exemplary device, according to the embodiments of the present disclosure.

FIG. 16 is an exemplary electronic circuit diagram to automate the PSA system, according to the embodiments of the present disclosure.

FIG. 17 illustrate the steps of pressure swing adsorption (PSA) in a two-column system, according to the embodiments of the present disclosure.

FIG. 18 graphically compares the weight % loading of zeolites and pressure across the zeolite bed.

### DETAILED DESCRIPTION

The embodiments of the present disclosure relate to an adaptive oxygen concentrator device. Specifically, the embodiments of the present disclosure relate to a smart oxygen concentrator that pairs with real-time oxygen titration. The smart oxygen concentrator device can detect and predict when the user or patient is idle or performing an activity that requires a ramp up or ramp down of oxygen supply. When such change in states is detected, the device will be able to automate the change in oxygen output settings, providing adequate amount of oxygen to the patient. In other embodiments, the device can adjust and factually change oxygen dosage based upon different activity levels of the patient. It is the first, true integrated oxygen device built ground-up.

It is also crucial that the device is able to ramp down oxygen supply for patients. As mentioned above, especially for patients with moderate/severe COPD whose target SpO₂ should be 88-92%, oversupplying oxygen to these patients will have adverse effects to their health, with potential risk of hypercapnic respiratory failure, which essentially means their respiratory system shuts down upon supply of overly concentrated oxygen, i.e., SpO₂ of 92-96%.

The device, in accordance with the embodiments of the present disclosure, can deliver medically equivalent concentrated oxygen with tailored actionable information that is smaller and lighter than current devices on market.

The device of the present disclosure may provide a true harmonization of digital healthcare and health hardware. The device of the present disclosure can revolutionize the oxygen industry that hasn't seen innovation in over a decade, making it the first adaptive device to cater towards personalized health as the smallest and lightest device on market that is designed and built for the world.

To date portable oxygen concentrators (POCs) currently available on market have no capabilities to:
use and record vital clinical data; and
change, adjust, and adapt oxygen intake based upon user's activity level.

Our primary research revealed insights from respiratory clinicians and users. Based upon on their professional experience with POCs as an oxygen treatment, specifically supplemental oxygen the following was noted:
Pulse flow on many of the POCs indicate that they can consistently produce a certain amount of LPM when, in fact, while functioning under pulse flow, if there is any increase in activity or breathing rate, the POCs often struggle to produce the desired amount of oxygen for the user.
As with any increase in breathing rate and volume demand, the filling of oxygen and purge of waste cycle do not keep up with the increased demand, and therefore are not producing the required LPM of oxygen for the user.

The device of the present disclosure comprises an adaptive oxygen concentrator device that can respond to the users' respiratory needs. The device may be intended to ‘smartly’ adjust, change, and adapt to the users' needs rather than the depending on manual input adjustments with its proprietary algorithm. To date, there is no portable adaptive oxygen device that has this ability and in turn prevent overdose and/or underdose of oxygen. The device of the present disclosure may be purposefully designed and built to ensure it caters to a wide range of individuals utilizing the device.

Clinicians have been reluctant to have patients adjust their own oxygen therapy devices. Key recommendations made by the Thoracic Society of Australia and New Zealand suggest the following:
In COPD patients and other conditions associated with chronic respiratory failure oxygen should be administered if the blood oxygen saturation level (SpO₂) is less than 88% and titrated to a target SpO₂ range of 88% to 92%.
In other acute medical conditions, oxygen should be administered if the SpO₂ is less than 92%, and titrated to a target SpO₂ range of 92% to 96%.

The ability to tailor oxygen flow to the needs of the patient is desirable in order to:
minimize episodes of low blood oxygen concentration (desaturation)
avoid excessive oxygen administration which can lead to respiratory acidosis
customize oxygen flow to patient needs, in particular during activity and sleep.

Automated oxygen titration benefits also include increased safety for patients, reduced time of desaturation and less potential for hyperoxia. A Canadian study used an automated closed-loop oxygen delivery system with the potential to optimize oxygen titration and reduce complications associated with the use of oxygen therapy. By providing a continuous monitoring within a closed loop system with the main parameter; SpO₂, a controller can be placed to adjust the oxygen flow with the aim of maintaining a predefined target of SpO₂ could substantially increase patient safety and physician and nurse adherence to corrected oxygen.

Further in some countries such as Australia, home oxygen prescription requires detailed instructions on the dosage oxygen levels for users. This level of detail will require oxygen equipment to be able to work within a range of lifestyles and activity levels. Users oxygen demands may increase and/or decrease throughout the disease progression. Flexibility of the device to change and adapt to these requirements would likely increase health quality of the user.

To date, current technologies are limited to their dose capabilities and oxygen purity, however the added ability to monitor oxygen saturation levels in real-time will be an indispensable to one's health. The data collected becomes crucial for physician who are looking to improve users overall health and quality of life.

The embodiments of the present disclosure provide an adaptive device that adds portability and the ability to tailor its algorithm to provide substantial benefits including, for example:
1) Decreasing the risk linked to desaturation whilst improved better clinical outcomes overall.
2) Corrected oxygen intake by linking traditional oxygen prescriptions with users activity levels
3) Providing physicians with more data revealing insights and patterns generally not picked up by an Arterial Blood Gas (ABG).

The device of the present disclosure offers a compelling eco-system paired with real-time oxygen titration allowing key feedback data between the users, clinicians and device, currently not seen in market-available devices.

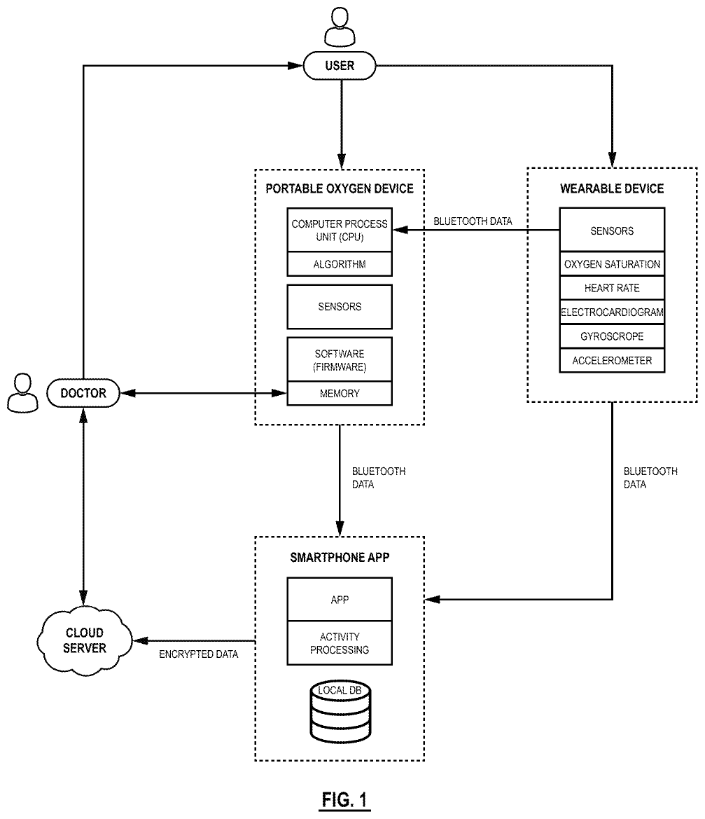

As illustrated in FIG. 1, the portable oxygen concentrator (POC) may store user health diagnostics. Practitioners and clinicians, for example “doctor” in FIG. 1, may be able to retrieve this stored user health data and prescribe better oxygen flow presets for the user. In other aspects, the POC may be able to connect to a data cloud server to upload and store the user health diagnostics. In another embodiment, the POC may be able to couple to various remote devices, including smart phone, computer, tablet, smart bands, or other wearable devices. The POC may connect to other remote devices wirelessly or via cables, such as a USB cable.

The POC may comprise a user interface configured to receive user input. The user input may be used to adjust the amount of oxygen released to the user. In some embodiments, the POC may comprise a wireless receiver in order to receive data from various remote devices. The remote devices may include, but are not limited to, a computer, a smartphone, or a wearable device.

The human body requires oxygen to be constant and continuous. Depending on your activity your muscles will work harder during increased activities and that means their demand for oxygen increases. This happens because oxygen is needed to burn calories more efficiently. Since the blood picks up oxygen in the lungs, and the demand for oxygen increases during exercise, the lungs must work harder. With a faster breathing rate, more oxygen is picked up at the lungs for delivery to the working muscles.

The body uses oxygen to produce energy, and this oxygen is supplied via your bloodstream. This results in a direct, positive relationship between your heart, breathing and physical activity rates. However, your physical activity rate can exceed your maximum heart and breathing rates. This results in the short-term production of energy without oxygen. By combining aerobic and anaerobic activities, you can greatly increase your strength, stamina, training gains and cardiorespiratory fitness.

Your heart rate, or pulse, is the number of times your heart beats in a minute. Depending on your age and level of physical fitness, a normal resting pulse ranges from 60 to 100 beats per minute. Your breathing rate is measured in a similar manner, with an average resting rate of 12 to 20 breaths per minute. Both your pulse and breathing rate increase with exercise, maintaining a ratio of approximately 1 breath for every 4 heartbeats.

Lung disease (also known as Chronic obstructive pulmonary disease (COPD)) or lung dysfunction in the breathing process means that an additional oxygen supply may be needed to meet the body's oxygen requirements. Normal oxygen levels are considered to be between 95-97% at sea level. The need for additional oxygen is determined by the level of oxygen in the bloodstream during rest, exertion and sleep. Oxygen levels below 90% indicate a need for oxygen supplementation in order for individuals to perform normal activities of daily life. This should be administered so as to minimize the potential of hyperoxia (excess oxygen in lungs or other body tissues) or hypoxemia (abnormally low oxygen in the blood). An oximeter (or Smart band) is used to tell how much of a person's blood is filled with oxygen. An SpO₂ (oxygen saturation level) oximetry reading can be used as a guide to tell how much oxygen is in the blood and what additional oxygen is required.

The device of the present disclosure connects these crucial physiological relationships together to create an adaptive algorithm capable of discerning a range of users' activity level and optimize oxygen flow to suite users' needs. The embodiments of the present disclosure provide a complete integrated system that may combine complementary electronics, adsorbents, and sensors designed for higher efficiency concentration of oxygen. The adsorbents may work with the system to concentrate ambient oxygen and generate an intended oxygen level. Multiple adsorbents may be utilized in a staged process to purify ambient air and increase oxygen purity output to reduce the volume of adsorbents, such as zeolites, required, thereby reducing the size of the device. In some embodiments, specific percentages of different adsorbents, such as zeolites, may be used in different layers to achieve a medical equivalent of concentrated oxygen. In some embodiments, the system may utilize sensor data, a formula, and/or an adaptive algorithm in order to adjust and change oxygen output near instantaneously with data readily available. The type of sensors required to drive the automation side may be linked with adaptive oxygen titration. In other embodiments, the system may determine the range of oxygen saturation needed to output the correct amount of LPM of oxygen. In some embodiments, the system may create an accurate reading of individual oxygen needs based on a digitalized adaptive algorithm that includes a range of primary (e.g., oxygen saturation) and secondary (e.g., breathes per minute, heart rate, respiratory rate) data readings of the user. The system may be used to minimize the overdose or underdose of oxygen. In some embodiments, data from the portable oxygen concentrator may be sent to a smart phone application to generate a report and allow the application to interact with the portable oxygen concentrator. The system may provide continuous flow and/or pulse flow of oxygen, and may comprise a controller that monitors oxygen and pressure output.

### Pressure Spring Adsorption (PSA)

Pressure Spring Adsorption (PSA) is a common gas separation method in chemical production plants, due to its simple and cost-effective nature in comparison to other medium to large scale separation methods. PSA is unique compared to other processes as whilst most other industrial separation processes operate under steady state, a PSA process is dynamic as conditions within the column are constantly changing. Eventually, the method may need to be scaled down in order to produce portable oxygen concentrators (POC), due to its great potential in mobility. The process operates within cycles in which a column repeatedly experiences a series of pressurization, adsorption, and regeneration steps.

Oxygen (O₂) is used in a variety of chemical processes and for medical purposes throughout the world. Current concentration methods are:
Cryogenic Distillation: This is the leading process for producing 99% of oxygen in bulk. However, this process needs a large amount of equipment, can be hazardous and energy inefficient.
Membrane Separation: For Medium to large scale production. However, this process requires a large surface area, large compressors and is a safety hazard.

Pressure Swing Adsorption (PSA): Use sorbents (zeolites, nanotubes) in two adsorption columns to separate molecules. The most common process uses two columns. However, in commercial industries, it will have four or more column systems. PSA has become an important alternative to cryogenic distillation and membrane separation for the concentration of O₂ from air with the commercialization of advanced adsorbents like zeolites.

PSA processes utilize a column packed with an adsorbent where feed mixture is introduced in one end of the column and the product exits the other end. The feed gas concentration changes with time within the column causing a concentration wave to form in the column as the adsorbate moves from the fluid phase into the adsorbed phase. This occurs in a mass transfer zone (MTZ) that travels through the column and eventually reaches the opposite end of the column. This results in what is called a breakthrough curve, which occurs when the outlet concentration of the adsorbate begins to increase, eventually reaching the inlet adsorbate concentration. The shape of this breakthrough curve is heavily dependent on the shape of the adsorption isotherm that exists between the adsorbent and adsorbate and whether the equilibrium is favorable or unfavorable for adsorption.

The basic premise of PSA involves one or more columns packed with an adsorbent (zeolite, carbon molecular sieve, etc.) which preferentially adsorbs one type of gas molecule compared to other in a gas mixture that passes through the column. This normally occurs at some pressure above atmospheric pressure until the gas nearly saturates the column with the more strongly adsorbed gas molecule.

The product is the gas molecule type that adsorbs less and comes out the product end of the column. In order to reuse the column later in the process, the undesirable components need to be removed from the column through desorption or regeneration. Desorption of the column is critical to the PSA processes.

Desorption in a PSA process occurs through changes in the pressure and composition of the column because they provide the quickest method of regeneration. Desorption occurs at either atmospheric or vacuum pressure causing the pressure to swing from high pressure during adsorption to a low pressure during desorption.

The overall efficiency of the device is described by the devices product purity, product recovery, and bed size factor (BSF). The selectivity of the adsorbent for a chemical species primarily determines the possible purity. Product recovery is a measure of how much desired component is in the high pressure product stream compared to the feed stream.

There is a trade-off between recovery and purity; i.e. high purity usually results in lower recovery. Maximum potential recovery is established by the affinity of the so!id for the heavy component over the Sight component through equilibrium. Recovery determines the energy efficiency of the process since it determines how much high pressure feed is utilized per product rate. The overall column design and the control philosophy and cycle time of the device will determine the BSF.

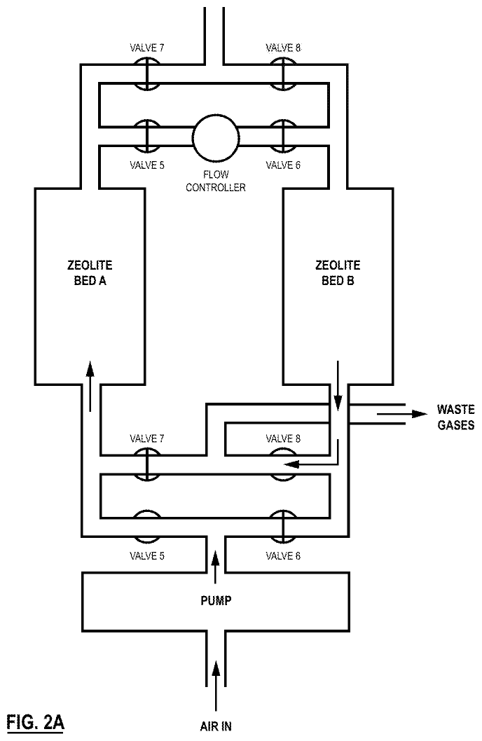

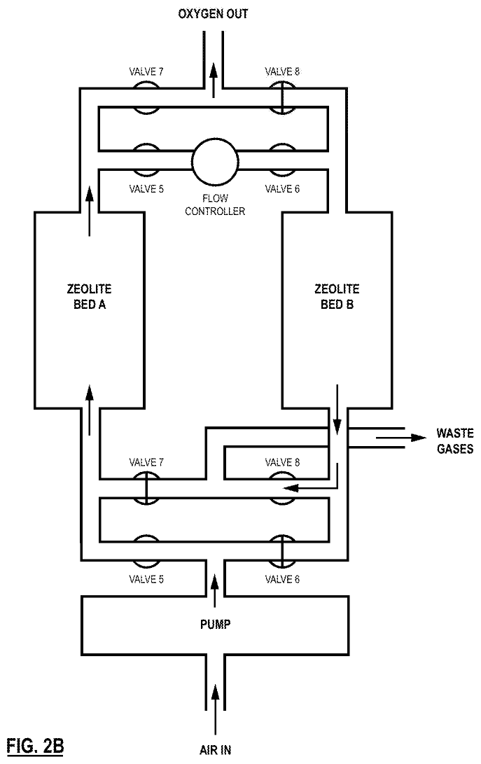

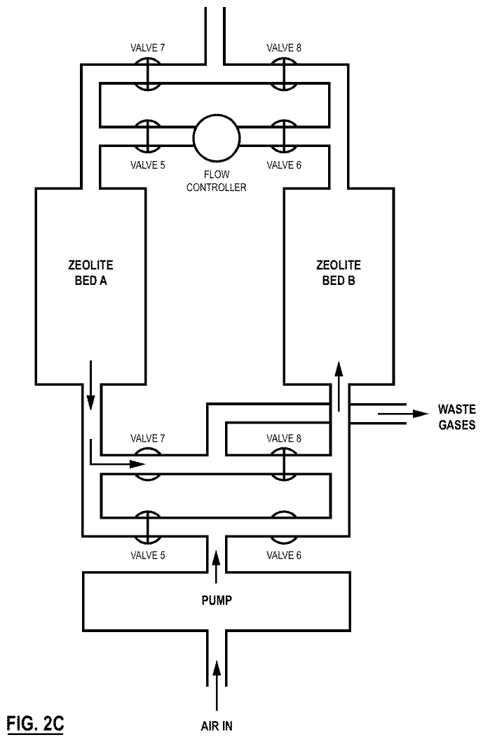

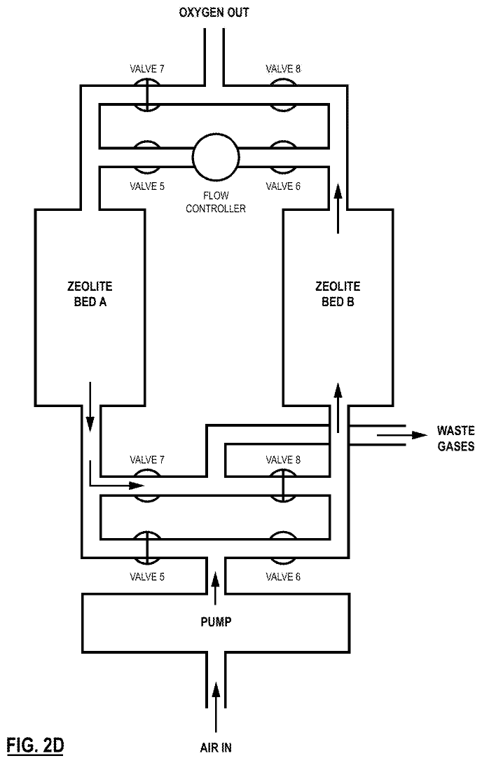

The device of the present disclosure uses the process of Pressure Swing Adsorption (PSA) with a combination of zeolites to obtain a concentrated level of oxygen suitable for a variety of uses including medical use. As seen in FIGS. 2A-2D the device of the present disclosure employs a two-column system design in a staged production and regeneration process. These steps are:
adsorption (adsorption)—FIG. 2A
production (pressurization)—FIG. 2B
blowdown (back purge); and—FIG. 2C p1 purge (desorption)—FIG. 2D

In FIG. 2A, compressed air is fed into zeolite bed A. Nitrogen and argon molecules are trapped in zeolite bed A, while oxygen is allowed to flow through zeolite bed A. In FIG. 2B, zeolites in zeolite bed A becomes saturated with nitrogen and argon molecules. The compressed air flow is then directed into zeolite bed B. In FIG. 2C, the zeolites in zeolite bed B absorbs nitrogen and argon molecules. Zeolite bed A is depressurized, thereby allowing argon and nitrogen molecules to be purged out of the system, e.g., “waste gases” in FIG. 2C, and released to the atmosphere. in FIG. 2D, the process starts over. Compressed air is once again fed into zeolite bed A, and zeolite bed B is depressurized, thereby releasing argon and nitrogen molecules in zeolite bed B out of the system and into the atmosphere.

Sequencing between each stage, as illustrated in FIGS. 2A-2D, is critical to the successful creation continuous oxygen production for PSA of the oxygen concentrator device. Between each stage, zeolites have a finite production of oxygen at one-time before it saturates and becomes exhausted. Being able to optimize and sequence the PSA flow, we are able to renew and regenerated the zeolites for continuous usage of zeolites.

### Device Design

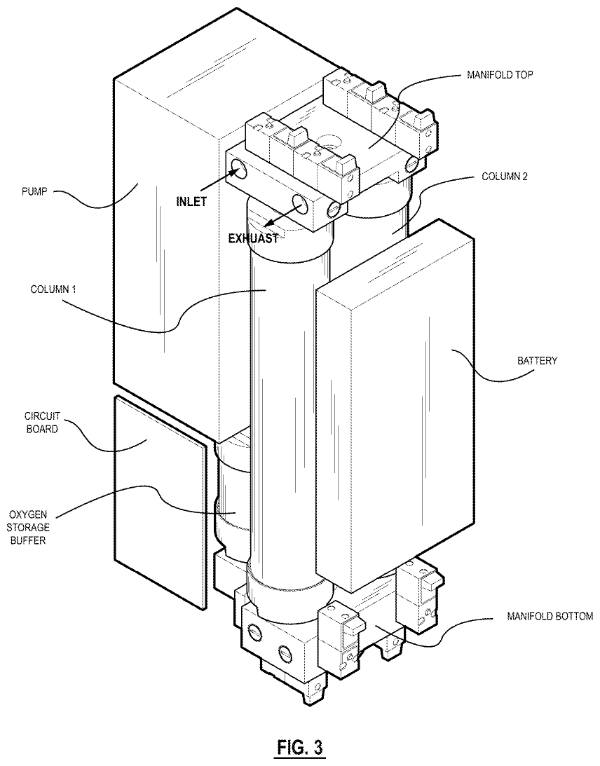

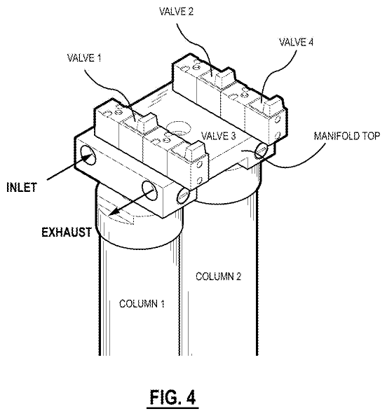

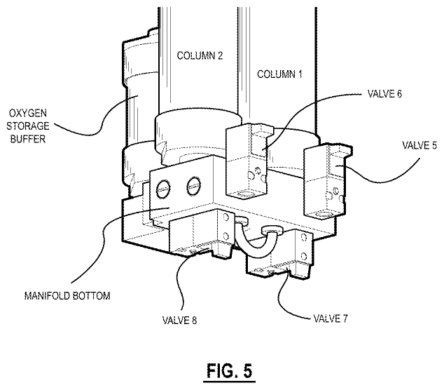

In FIGS. 3-5, an exemplary device in accordance with the present disclosure is provided. The technology package implemented in the device of the present disclosure is built specifically to reduce size and increase modularity between each component. The top and bottom manifolds, e.g., manifold top in FIGS. 3 and 4, and manifold bottom in FIGS. 3 and 5, integrate with columns 1 and 2 by solenoid valves that sit on each end of columns 1 and 2. A compressor (not shown) that is capable of delivering freeflow air at about 5 liters per minute (LPM) to about 15 LPM may be used. In an embodiment, the compressor is capable of delivering freeflow air at about 6 LPM to about 12 LPM. The compressor may have a capacity between about 1 bar to about 5 bar pressure, or preferably between about 1.6 bar (about 14 psi) to about 2 bar pressure (about 28 psi). In another embodiment, the compressor may have a capacity of about 1.4 bar pressure (about 20 psi) and may be capable of delivering freeflow air about 1.4 LPM to about 3.3 LPM. In some embodiments, the compressor is connected to the rest of the manifold design via a plastic tube, pushing air into the vessel/columns, e.g., columns 1 and 2 in FIGS. 3-5, thereby allowing PSA exchange to happen.

The vessel/column design encompasses a number of unique attributes and is a customized vessel/column to hold zeolites. The design of the columns (zeolite housing) had to follow a few key points for them to operate properly. First, the columns, e.g., columns 1 and 2 in FIGS. 3-5, needed a sealed structure up to or exceeding the pressure that is required. The columns also had to reduce air flow drag with an unobstructed air flow path. In addition, the columns needed a means for letting air through under pressure and keeping zeolites inside. Lastly, the columns needed to compress the zeolites and keep them compressed in order to reduce zeolite movement and vibration.

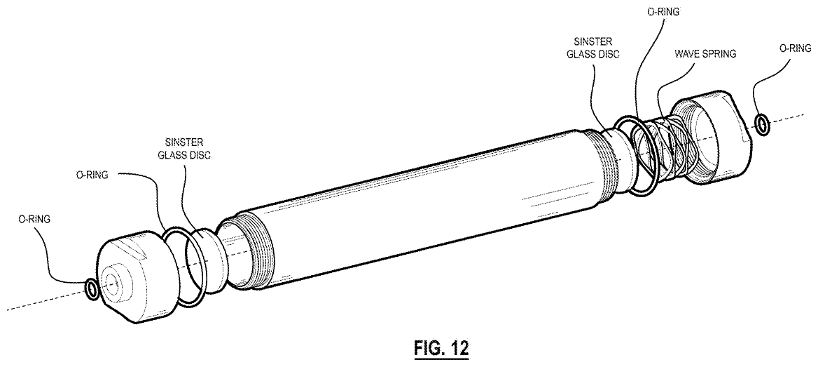

First and foremost, a sealed structure may be made of aluminum for ease of manufacture, and may be coupled with O-rings (for example, O-rings in FIG. 12) to seal the structure. Not only may this improve results with oxygen production, but it also may keep the zeolites within the columns from absorbing nitrogen from the surrounding air when not in use.

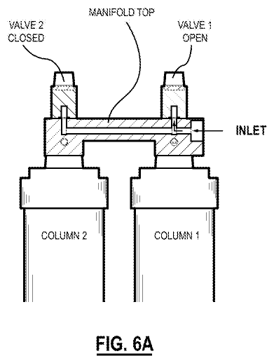

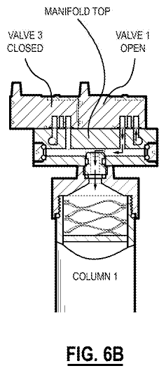

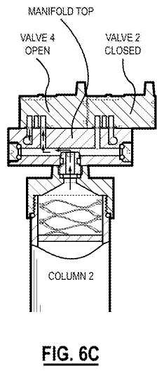

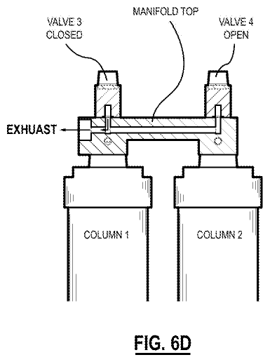

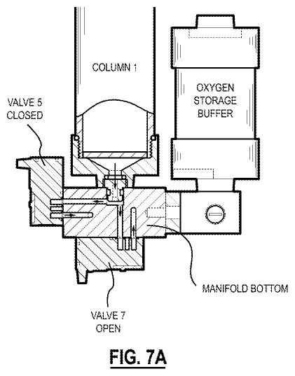

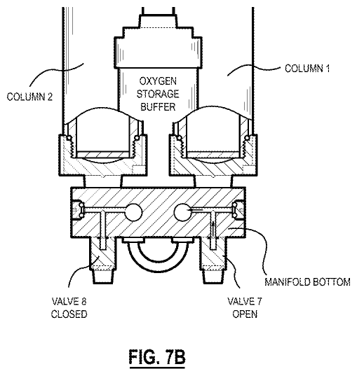

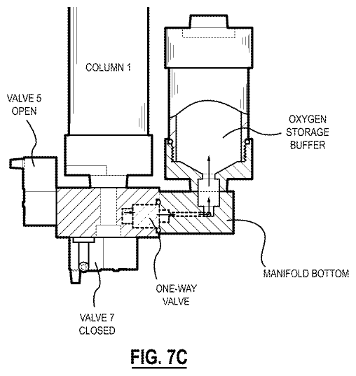

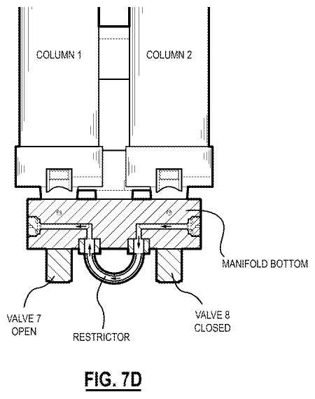

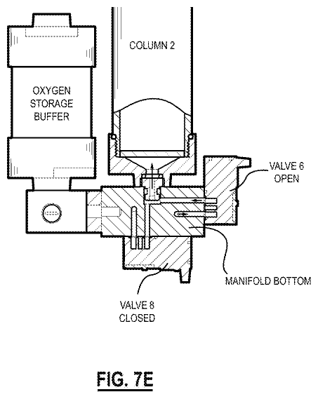

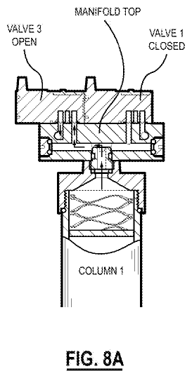

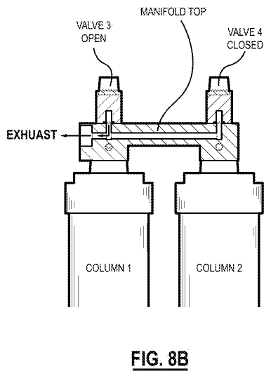

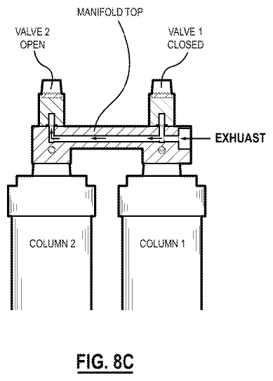

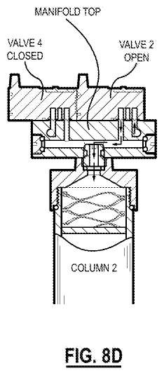

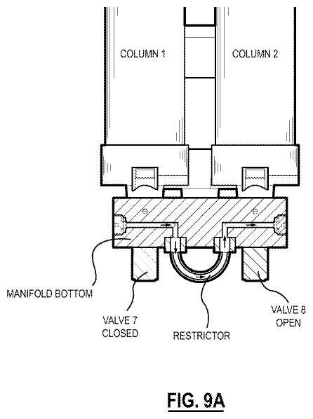

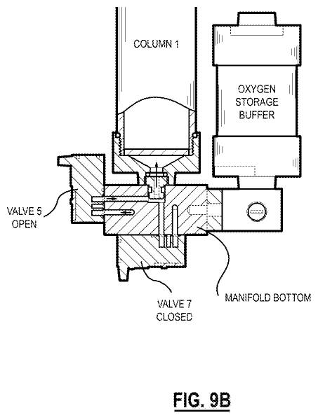

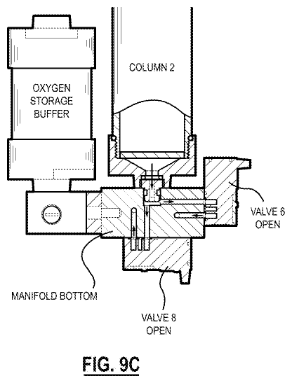

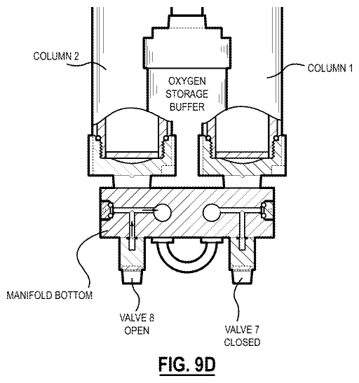

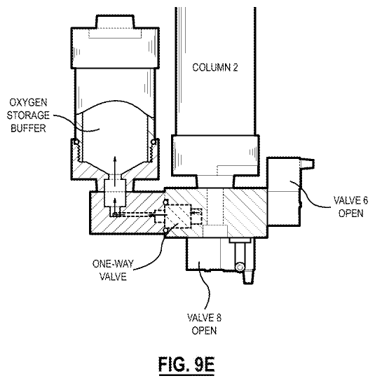

Reducing drag on air flow in and out of the columns has proven to be very important in reaching higher production levels of oxygen. As seen in FIGS. 6B, 6C, the caps that close off each end of columns 1 and 2 have been designed with tapered flow paths to help direct air in and out of columns 1 and 2. Altering the flow path in this way can help reduce cycle times of the column and heat in the mechanical parts. In our development we have noticed that heat has directly affected oxygen production reducing our capabilities to be efficient with each cycle.

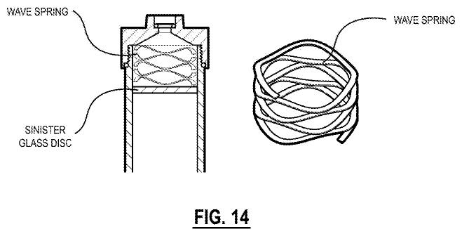

Furthermore, filtering zeolites from the compressed air could be done with sintered glass filter discs (for example, sintered glass disc in FIGS. 12 and 14) as their rigidity and porous features were perfect for the application. Filtering serves several purposes, including cleaning ambient air, preventing large microbes from entering the system and contaminating the purity of the oxygen, preventing moisture from entering the columns and permeating the zeolites which in turn affects the performance of the PSA system. Air needs to be as dry as possible in order to maximize the effectiveness of the zeolites. The sintered glass filter discs may also prevent the zeolites from escaping the columns. The device may comprise one or more filters. In one embodiment, the device may comprise a filter before air enters the compressor. Silica gel may be used as the filter. In addition, the device may comprise another filter before air enters the columns, such as a sintered glass disc filter disposed inside the columns.

In addition, compressing the zeolites in the columns is fundamental. Alternative designs of the filtration system may comprise dense foam material open cell or rubber durometer. The dense foam material may be a viable option to be used as a substitute that acts both as a filter and a wave spring. Rubber durometer, for example, may have a shore grade of 30A to 4QA. By providing no room for the zeolites to dodge the air moving around them at speed the zeolites were forced to perform as they are intended to. As illustrated in FIGS. 12 and 14, for example, the columns have been designed in such a way that a wave spring can be inserted in between the cap's underside and the sintered glass filter disc mentioned above. The spring is compressed as the cap is screwed down forcing the sintered glass disc to push down on the zeolites below.

The columns that holds the zeolites may be custom made in a singular aluminum block through computer numerical control (CNC) routing. For mass production, the design of the columns may use the latest engineered thermoplastics (e.g., Polycarbonate/ABS), and the columns may be vacuum formed. The columns may be formed at once saving space and may have a common wall to both adsorbent columns. The device requires relatively low pressures and temperatures and hence an engineered thermoplastic material can be used. All devices on the market at the moment use either machined or rolled aluminum. In addition, the columns may include a unique manifolding system at the top of the column to improve the overall space efficiency of the design. The column design may be modular and may allow extra capacity of zeolites to be loaded as a cartridge when required during peak exercise allowing the size of the device to be flexible depending on the user's specific activity (just like loading an extra batter. Moreover, the column design may include an integrated dual column that has a common wall, i.e. two pressure columns in one overall column assembly. The dual column design may be made of extruded thermoplastic and may reduce the overall space required for the overall assembly.

In some embodiments, the columns may vary in shape. For example, the columns may be cylindrical, rectangular, and/or triangular in shape.

In some embodiments, the top and bottom manifolds may be injection molded with aluminum. In other embodiments, various components of the device may be manufactured via casting with finishing processes, 3D printing in metal with finishing processes, or 3D printing in wax or plastic for casting. For example, the columns may be manufactured via 3D printing. In other aspects, the device may be casted using aluminum in order to efficiently create the finer details of the device.

PSA processes utilize a column packed with an adsorbent where feed mixture is introduced in one end of the column and product exits the other end. The feed gas concentration changes with time within the column causing a concentration wave to form in the column as the adsorbent moves from the fluid phase into the adsorbed phase.

PSA uses one or more columns packed with an adsorbent (e.g., LiLSX zeolites, 5A Zeolites and etc.) which preferentially adsorbs one type of gas molecule compared to others in a gas mixture that passes through the column. This normally occurs at a certain atmospheric pressure until the gas saturates the column with the more strongly adsorbed gas molecule. Desorption of the column is critical to the efficiency of the process and is a step where improvements are made to increase the extent of regeneration in order to maximize the removal of the heavy component and increase the efficiency of the process.

In FIGS. 4 and 5, an exemplary top manifold and an exemplary bottom manifold according to the embodiments of the present disclosure are provided, respectively. The manifold designs of the device may contain internal tunnel networks specifically milled at particular points where the miniaturized solenoid valves sits. The solenoid valves are electromechanically operated valves and they are controlled by an electric current through a solenoid in the case of a two-port valve the flow is switched on or off. This allows air to flow between each vessel/column without the need of extra tubes. In one embodiment, the solenoid valves may be controlled by an Arduino board, which is described in further detail below. The Arduino board may be programmed to control the opening and closing sequence of the solenoid valves in the device. In other embodiments, the solenoid valves may be controlled by other hardware or software programs, including small board computers like a Raspberry Pi.

In addition, manifolds have been designed in such a way to provide minimum loss of pressure over the system, as we are still experimenting the manifolds have been designed with redundancies offering the ability to reduce/alter hole orifice size. This feature helps us fine tune the systems output, exhaust and flow to be more efficient. The diameter of the holes on the top manifold and the bottom manifold may vary in diameter. In some embodiments, the diameter of the holes may be about 3 mm on the top manifold and about 1.5 mm on the bottom manifold. In other aspects, the ratio of the diameter of the holes on the top manifold to the diameter of holes on the bottom manifold may be about 2:1 . In other aspects, the ratio of the diameters of the holes may be adjusted to reduce the risk of a pressure drop and to allow for better breathing throughout the device.

The manifold may be made from aluminum for its lightweight and ease of manufacturing features. O-rings may be added to help seal holes more reliably making for a leak free test rig. One way shut off valves, called check valves, designed for taps may be inserted into the system to help stop the system from flowing backwards. Furthermore, O-rings in combination with the check valves can be inserted into the system, especially in the manifold to ensure secure sealing.

As well as integrating the manifold designs with the column, before ambient air enters the vessel/column it is important to have dry and oil free air. Therefore, a compressor design that is oil free in it is overall design may be used. Compressors that do not require oil for the lubrication and sealing are advantageous as this does not introduce any additional contaminants to the zeolite media which would otherwise contaminate and reduce the efficacy of the zeolite media to adsorb and reduce the overall operating efficiency of the device.

A pre-set amount of Alumina Zeolites is layered on top of the LiLSX zeolites to remove any moisture from the incoming ambient air. The pre-set amount of activated Alumina Zeolites may be determined by the following formula:

$$Weight Ratio \frac{Activated Alumina}{LiLSX Zeolite} = 0.2 to 0.5$$

Moisture in ambient air has two effects of reducing the overall compressor efficiency and also contaminating the zeolites themselves. The zeolites work the most efficient when dry clean air is passed through it. The ‘recipe’ is designed to sacrificially remove water vapor from the air. For example, the oxygen concentrator may use an activated alumina composition in order to remove water molecules from air before air reaches the zeolites. Otherwise, the water molecules may be adsorbed by the zeolites instead of nitrogen, thereby impeding zeolite performance.

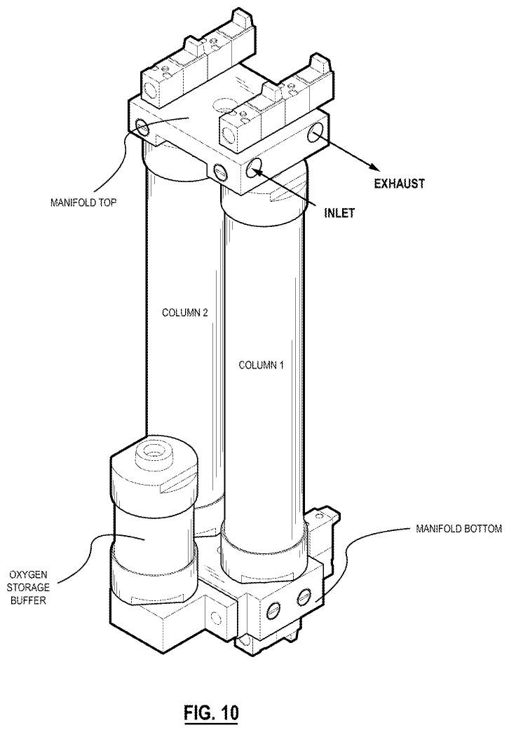

In FIG. 10, a smaller version of the vessel/column is located upstream of the device and acts as both an oxygen storage buffer to contain about 90-93% medically equivalent oxygen. In a continuous oxygen feed setting, where oxygen is constantly produce without interruption, the oxygen storage buffer's main functionality is to provide oxygen during those down peaks.

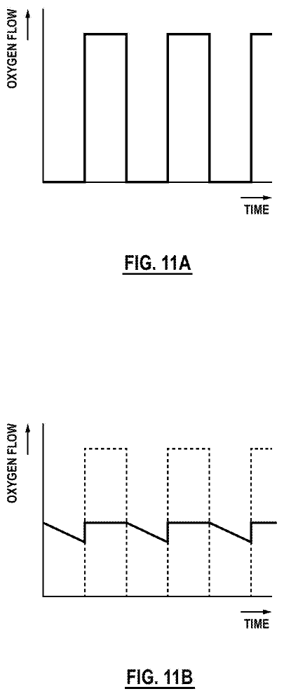

Current POCs devices are unable to provide to continuous flow due to technological limitations at time of their development. As seen in FIG. 11A, current POCs have fixed pulse flow to reduce size and increase battery length. This pulse flow delivers oxygen every time the patient takes a breath using a sensor to sense change at the output of oxygen from the device. If their breathing rate increases, the machine will generate an alarm. Unfortunately, this is dependent on the sensitivity of the sensor which means that the device may not output the oxygen. Additionally, the oxygen output would be at a linear set amount, meaning over dosage and/or under dosage of oxygen is a high possibility.

The device of the present disclosure is developed from ground-up with users and doctors in mind. The device may utilize its adaptive algorithm in the background to provide an accurate gauge on the user's activity level and output out the corrected amount of oxygen to suite. Doing so, as seen in FIG. 11B, the device may be able to switch between pulse flow and continuous flow on-demand through either the input of the adaptive algorithm or manual input by the users. This is an adaptive model, rather than a reactive model. The device will change according to the users' physiology rather than using manual input.

In FIG. 12, an exemplary column design of the device according to the present disclosure is provided. The length to diameter ratio is important to the overall efficiency of the operation of the device. In some embodiments, the diameter of the tube could be small and the length of the column could be long. This may maximize contact surface area for zeolites to adsorb ambient air without having significant pressure loss across the length of the column. Therefore, this may maximize the overall efficiency of the adsorbent media and eliminate any dead spaces. In other embodiments, the diameter of the tube could be large and the length of the column could be short.

Each column may have an equal or equivalent amount of grams of zeolites in order to have consistent airflow throughout the column and PSA system to operate effectively. If the two columns are uneven in grams, the PSA system will immediately have pressure drop as the air flow across the columns will be different, the cycles will be different and the oxygen output will decrease dramatically.

The diameter to length ratio may be about 1:6, thereby equaling approximately 26 mm in diameter and about 178 mm in length. Proportionally, 1:6 ratio sizing will allow the total weight of about 55 grams of zeolites to be used per column. However, in some embodiments, the columns may each comprise between about 20 grams and about 80 grams of zeolites.

### Radial Flow/Pathways

Conventional PSA generally has an axial flow configuration characterized by ratio of bed length to bed diameter, L/D>1 and L/D<1, for vertical and horizontal packed beds, respectively. Changing the packed column configuration to a radial flow geometry may give comparable performance to that of axial bed, and the radial design may also offer additional benefits of large cross-sectional area, small pressure drop and ease of scaling up. Unlike the conventional axial flow configuration, the radial design may implement a radial flow configuration, in which air is channeled in a radial flow direction across the zeolite beds.

For example, the radial design may increase interstitial flow velocity toward the center and sharpen the concentration wavefront, thus promoting deeper feed penetration and resulting in higher adsorbent utilization. In addition, with a radial design, purge gas flows radially from inner toward outside cylinder. The large exposure of the outside cylinder to low pressure promotes depressurization and desorption. Furthermore, for the same feed pressure and same amount of adsorbent, the separation performance of radial flow PSA is better than that of the axial flow PSA by utilizing smaller particle size. Particles as small as a few pm could be used directly due to the large cross-sectional area that lowers the pressure drop. Smaller particle size facilitates faster adsorption kinetics and enables rapid PSA. Moreover, while thicker radial bed is better in term of lower pressure drop because it offers larger cross-sectional flow area, planar radial bed is better in term of higher heat transfer rate because it offers larger planar surface area. Both features are important for radial bed because it may be used to process large flow rate that brings up high pressure drop and heat excursion problems. Additionally, zeolites packed in the radial beds may be exposed to a larger quantity of air, thereby reducing the pressure drop and increasing zeolite utilization within the same or similar volumetric space as that of the conventional axial flow configuration. Because the radial design may increase zeolite utilization, the radial design may also decrease the total amount (in grams) of zeolites required to produce oxygen. The radial design may decrease the amount of air required to operate the overall system, thereby reducing the compressor requirements, the weight of the battery, and the overall size of the POCs.

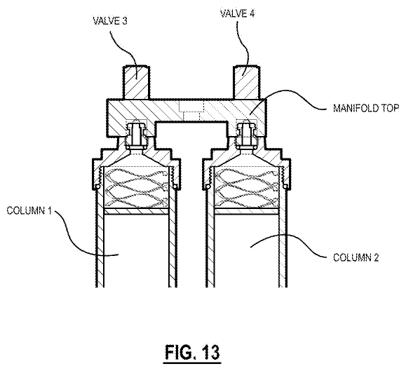

FIG. 13 and the figure above illustrate the tapered-funnel. As seen in the figures, the tapered-funnel is at the top, and the blue arrows show the inlet and out of compressed ambient air to the vessel. The red arrows indicate the flow of compressed ambient air at the interface between the lid and the packed zeolites. The wave spring is used to compress the sintered glass disc down on the packed zeolites.

The device of the present disclosure utilizes a customized lid design that incorporates a hybrid tapered-funnel that houses a spring. Flat springs are utilized to save space and not compromise on the compressive strength of the spring. The lid has a tapered funnel integrated into the lid design to channel the flow in and out of the columns. This minimizes short circuiting in the zeolites column and directs the flow evenly onto the loading surface of the column.

During PSA operation the zeolites expand and contract depending on the stage of operations. Maintaining a steady flow allows minimization of any fluidization of the zeolite bed. As seen in FIG. 14, the design of the device of the present disclosure incorporates a wave spring with a sintered glass disc, keeping the media under a variable compression (via a spring) allows approximately the same amount of compressive force on the zeolite media keeping it packed and minimizing any fluidization of the media bed. The wave spring may be disposed between the sintered glass disc and a cap of the column in order to create a cavity that the spring can compress and contract with no zeolites in it. The wave spring allows the sintered glass disc to move up and down during the compression and decompression of the columns, thereby providing the ability to compress the zeolites down over a period of time as the zeolites vibrate into the most efficient compressed state possible. The wave spring and the sintered glass disc function together to minimize gaps between the zeolites, thereby letting air move through more efficient paths in the column and preventing zeolites from moving out of the way of moving air.

The sintered glass disc is held in place by plastic structure which also acts a pinpoint to hold the wave springs. The sintered glass disc is a finely porous glass, allowing filtration of ambient air down to its nanometer preventing any zeolites leaving the column. The wave spring is used as it requires less travel distance in the longitudinal length of the spring for the equivalent compressive strength in comparison to a helical compression type spring. An O-Ring is used to air-seal the design.

A sintered glass disc may be used both at the top of the vessel/column where the wave spring is placed and also at the bottom of the column acting as a separator and filter paper all in one.

This unique combination of sintered glass disc and wave spring provides the following benefits:
a) At the lid interface an exact opening is cut to match the outer diameter of the wave spring to ensure that the spring seats into the lid of the device
b) At the zeolite interface a wave spring assembly has been designed that allows the travel of the spring and the fritted glass media up and down to ensure an even compression of the zeolites during all stages of the adsorption PSA process.

By utilizing these techniques, the device of the present disclosure is able to shrink the size of the overall device by utilizing micro-millimeter spaces to maximize functional output and minimize the size of the device.

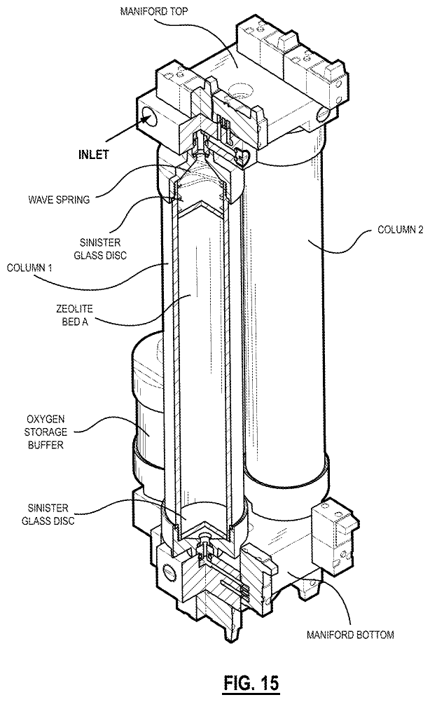

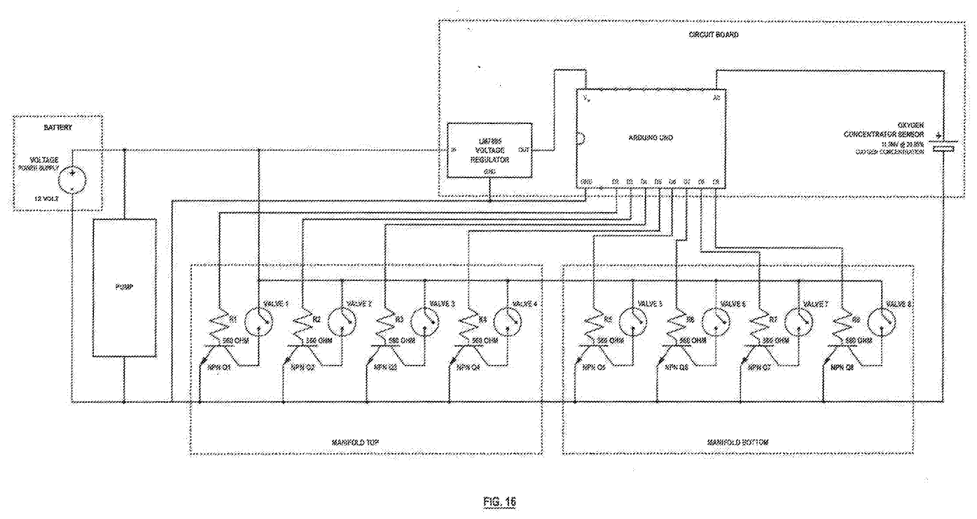

In FIG. 15, an exemplary manifold unibody design of the device according to the present disclosure is provided. The manifold unibody design of the device is a single structure design. This unique combination allows for a lighter and more rigid frame, allowing for increase in durability and a decrease in the amount of components required. Additionally, unique to the design of device is the integrated group of internal tubes within the unibody structure. At each outlet of device's manifold design, there is a 2/2 way electronic solenoid valve linking to a different set of tubes, pushing ‘cleaned’ air from Column 1 to Column 2. The valves mechanically restrict air flow between the inlet and the outlet. However, when the solenoid is energized by electrical current, the solenoid magnetizes and lifts open the valve, thereby allowing the air to flow through from inlet to outlet. This minimizes external surface area and dramatically decreases the amount of components required to link each valve together.

In order to deliver pure oxygen to the user, the device may comprise an output or an oxygen nib that allows the patient to connect the output of the device to a nasal prong, face mask, or equivalent thereof to inhale the oxygen.

### Electronic Circuit

Electrical and electronics design is critical to the automation of PSA systems, as electrical power is converted into energy to separate air. In FIG. 16, an overview of an exemplary electronic circuit is shown.

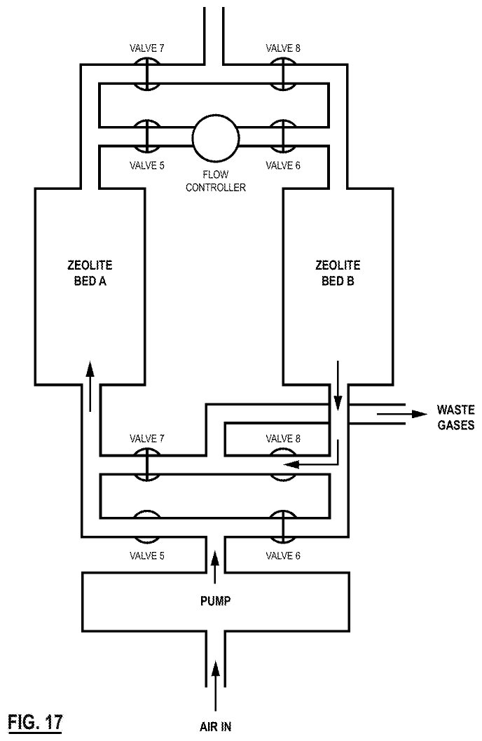

An electronics platform (such as an Arduino board) may be programmed to control the valves in a 4 stage PSA sequence as shown in FIG. 17. The 4 stage PSA sequence is also illustrated in the table below.

Each stage may be a sequence, and the timings for each stage may be as follows:
Stage 1: 8.0 seconds
Stage 2:0.1 seconds
Stage 3:8.0 seconds
Stage 4: 0.1 seconds

The timings for each stage may be adjusted to vary the amount of oxygen delivered to the user. In other embodiments, the timings within each stage may further be adjusted to increase or decrease cycle time within for faster PSA operations. In turn, adjusting the cycle time within may affect how quickly the device may start up.

The amount of oxygen delivered to the user may be dependent on the size of the columns. For example, the amount of oxygen delivered may depend on the length and diameter of the columns, weight ratio, as well as how the ambient air moves within the column. Therefore, the column may be “axial” in design, meaning air may only travel vertically up and down the column. This way, the device may only be capable of outputting a predetermined amount of oxygen to the user. For instance, in order to increase the amount of oxygen produced, the amount of zeolites used may need to be increased.

In other embodiments, the column may be “radial” in design, meaning air may travel vertically and/or horizontally, up and down the column, and left and right of the column. This way, the same amount of ambient air, if not smaller amount of ambient air, may be channeled through the columns and increase contact with the zeolites in the zeolite bed. This could significantly increase the amount of oxygen delivered to the user at a much smaller size, i.e. length, diameter, and weight ratio of the columns.

### Adaptive Algorithm

A proportion-integral-derivative (PID) controller may be used to apply accurate and optimal control. The overall control variable is the output of the device in terms of LPM of O₂ which will be governed by the flow rate of the device altered by a variable speed DC drive brushless motor. The speed of the motor may be varied to match the desired output of oxygen. The variables motor speed may be defined by the primary variable SpO₂ and trimmed by secondary variables such as heartbeat, respiratory rate, and/or flow rate.

Other feedback mechanisms will be certain alarms (high, low etc) to detect certain conditions and will trigger certain actions. Alarms will generally be:
High flow
Low flow
High pressure
Low pressure
High O₂ concentration
Low O₂ concentration
Number of run hours

In some aspects, the alarm may be an audible alarm that is triggered to alert people nearby to aid the user. In other aspects, the alarm may be a visual alarm. For example, the device may comprise an LCD or OLED display that is configured to display a visual indication. The display may further display information on the presets the user selects. The display may further assist in troubleshooting of the device.

The adaptive algorithm of the device may take in digital data from a number of inputs and make adjustments to match the oxygen demand requirements.

The device of the present disclosure may comprise sensors to collect a real-time feed of patient data on physiological parameters, including but not limited to volume of air breath, CO₂ concentration in air exhaled, SpO₂-O₂ Saturation in blood, heart rate, pulse rate, average breaths per minute, inhale pressure, exhale pressure, or sound of breath.

The parameter(s) measured above may be analyzed by the device and an adaptive algorithm may be applied to the data to identify the live health status of the patient. With this health status, the device may be able to predict and adapt to the changes in a patient's activity. As such, the adaptive algorithm of the device becomes ‘smarter’ and ‘adaptive’, tailoring itself to individual users. The accuracy of each measurement may be based upon third parties ability to gain regulatory approval such as FDA approval and thereby determine how accurate the device's adaptive algorithm could become. The device's adaptive algorithm can be further pre-set by physicians within a minimum and maximum capability, whilst patients can still determine whether or not they require the adaptive functionality. The device may be used in a wide range of markets, including but not limited to, chronic obstructive pulmonary disease (COPD), asthma, pneumonia, heart failure and chronic bronchitis.

### Oxygen Saturation (SpO₂)

Oxygen saturation is a percentage measurement of patients circulating hemoglobin combined with oxygen. A pulse oximetry is generally used to determine non-invasively SpO₂and provides continuous monitoring of oxygenation state. A pulse oximetry can be a useful guide to measure desaturation (low blood oxygen) that may occur during activity such as exercise, a drop of at least 4% below 90%. This would be a primary indicator of low oxygen and signal an increase in oxygen production requirement for the patient. The average healthy person should have SpO₂ of 94% to 99%. For users with mild respiratory disease SpO₂ should generally be 90% to 94%. Normally, more than 89% of the body's red blood cells should be carrying oxygen. An oxygen saturation level of at least 89% keeps body cells healthy but if low oxygen levels occur too frequently body cells can be strained or damaged. An adjustment of oxygen flow within a range would aim to maintain SpO₂ within a predefined target by the physician.

As pulse oximeters have become more readily available over the last few years, it is a known fact that these types of technology will secure itself into the next generation of wearables and smartwatches like the Apple Watch pending FDA approvals. To run the adaptive device, the device's adaptive algorithm relies on third party SpO₂ to be the primary controlling variable for its operation. If the SpO₂ is high or normal, then the device will adjust itself to save power and if a low SpO₂ was detected then this would be a key indicator of low oxygen and signal an increase in oxygen production requirements to the user.

Respiratory Rate (BPM)

The respiratory rate is the number of breaths a person takes per minute. In the device of the present disclosure, the respiratory rate will be measured by the increase/decrease of positive/negative pressure between oxygen tube and the oxygen outlet nib. This increase/decrease of positive/negative pressure signals is converted to a numerical number and compared against time to get ‘Breathes Per Minute’.

The normal respiration rate for an adult at rest is 12 to 16 breaths per minute. A respiration rate for an adult of under 12 breaths or over 20 breaths per minute while resting is considered abnormal. The respiratory rate is one of the four main vital signs of the human body along with body temperature, blood pressure and pulse.

Respiratory rate is a secondary measure of the user's well-being and activity level. An abnormal respiratory rate is a predictor of potentially serious clinical events. Ventilation is driven by both the arterial pressure of oxygen (PaO₂) and the arterial partial pressure of carbon dioxide (PaCO₂), with PaCO₂ being the most important driver. The body attempts to correct hypoxemia (low concentration of oxygen in the blood) and hypercapnia (carbon dioxide retention in the blood) by increasing both tidal volume (the amount of air entering lungs during normal inhalation at rest) and respiratory rate. Thus, these conditions can be detected by measuring the respiratory rate.

A higher respiratory rate potentially indicates a higher level of activity and this would be a contributing signal to either have the device of the present disclosure ramp up or down. Measuring respiratory rate directly is challenging and our device uses a surrogate pressure sensing transmitter on board the device, on the outlet of the oxygen output, to detect when the user is breathing the oxygen.

Tailoring oxygen to the needs of patients is not only desirable but necessary for effective results.

Adjustments in oxygen flow should therefore meet several objectives, including but not limited to, minimizing episodes of desaturation, avoiding excessive oxygen administration, and customizing the oxygen flow to the patient's needs.

Respiratory rate may be used in the device's adaptive algorithm as a secondary measure of the user's wellbeing and activity level. The higher the respiratory rate, indicates potentially a higher level of activity and this would be a contributing signal to either have the device ramp up or down. Measuring respiratory rate directly is challenging, and our device uses a surrogate pressure sensing transmitter on board of the device on the outlet of the O₂ output to detect when the user is breathing the oxygen.

Pulse Rate

The pulse is a direct measure of heart rate. A normal adult resting pulse is between 60-100 heartbeats per minute. A pulse oximeter can also be used a light-emitting diode (LED) and a photodetector to estimate the percentage of total hemoglobin that is saturated with oxygen, based on the amounts of red and infrared light that pass through the vascular bed. However, relying on pulse oximetry alone for adjusting oxygen supply could compromise patient safety.

Pulse oximetry can inform about saturation only. To be most effective it must be used in conjunction with monitoring of the patient's respiratory rate.

in the device's adaptive algorithm, the pulse rate can be a surrogate measure of the overall well-being of the user and can also determine whether or not a user is active or not. For example: A higher pulse rate would indicate a higher activity level and if combined with detection of a higher respiratory rate and SpO₂ as well, it would be a key indicator for the device to increase in oxygen output to match the users oxygen requirements.

Flow sensors are installed on board the oxygen concentration device to measure the production purity of oxygen. The flow sensors provide feedback to the device that the correct amount of product is being dosed to the user. Any out of expectation values may trigger an alarm or fault, thereby prompting the user to action.

The oxygen sensor is a primary sensor to monitor the production purity of oxygen. This device is important to validate that the desired concentration of oxygen is correctly being dosed for the user. If the value is either too high or too low, the device may issue an alarm/fault, thereby prompting the user to action.

### Adaptive Oxygen Titration

Oxygen therapy can be life saving for patients, especially those with chronic obstructive pulmonary disease (COPD) and is the backbone of any acute COPD treatment strategy. Oxygen should be considered as a drug that is prescribed and administered for specific indications, with a documented target oxygen range, and with regular monitoring of the patient's response.

Oxygen therapy is largely considered to be a benign drug. Since 1949, it has been consistently highlighted the need to accurately adjust oxygen delivery, avoid the risks of hyperoxia, and inducement of hypercapnia. Recent clinical data has shown excess oxygen may not entirely be good for the human body. For example users inhaling excess amounts of oxygen can lead to an increase in change of carbon dioxide levels potentially leading to carbon dioxide poisoning. With COPD patients, excess oxygen may have the adverse opposite affect where the patient is not exhaling enough to relieve carbon dioxide build up and are instead retaining it, leaving their lungs in worse shape than before the oxygen treatment. During daily activities as well, arterial oxygen desaturation is also common among COPD patient.

Oxygen flow for these patients is usually set at fixed and low rates for ambulatory patients. The adjustment of oxygen flow within a range would aim to maintain SpO₂ within a predefined target that can be determined by the physician in charge.

The ability to tailor oxygen therapy dependent on user activities is highly desirable. Therefore, adjustments in oxygen flow should meet several objectives, including but not limited to, minimizing episodes of desaturation, avoiding excessive oxygen administration that may be responsible for respiratory acidosis, and customizing the oxygen flow to patient's needs, especially during activity.

The device's adaptive oxygen titration is based upon three sets of available data, which are SpO₂, Respiratory/Breathe Rate and Pulse/Heart Rate. The adaptive algorithm that allows this to happen can be calibrated as more data is included.

In one embodiment, to enable the adaptive oxygen titration to function across the device of the present disclosure, the device may be sized for a maximum flow rate. Normally the design capacity does not operate at its maximum limit. The device may be sized for 5 LPM O₂ output as 100% capacity. Typically at rest, it may operate at 3 LPM, and at peak exercise times, it may increase the speed of the device to increase the output of the compressor to make the extra capacity. The volume of adsorbent media and the size of the compressor may be sized for the maximum output. Battery may be sized for the average use. Battery sizing strategy may be for 8 hours or average use or the shorter 4 hours for the peak usage should the user need to do exercise. Battery may comprise removable lithium-ion batteries with an external adapter to charge the battery. The batteries can be replaced for additional run time on-the-go.

In another embodiment, to enable the adaptive oxygen titration to function across the device of the present disclosure, modular design of the device may be sized for a maximum flow rate at average flow, e.g. 3 LPM O₂, production. An additional cartridge may be added to boost the capacity of the system to 5 LPM to deliver the extra oxygen at extra capacity. Compressor may be sized for the larger flow rate. Battery is sized for the average use.

The key sizing capacity is the LPM of O₂ production which is calibrated to the clinical set points SpO₂, BPM, heart beat etc.

### Zeolite Recipe

To produce medical equivalent oxygen, the device may use a zeolite recipe comprised of 5A zeolite and LiLSX zeolites or comprised of Alumina zeolite and LiLSX zeolites. Optionally, AgX zeolites may also be added.

World Health Organization (WHO) recommends oxygen concentrators to be an “effective means of supplying oxygen”. In Canada, as many as 48 hospitals reported safe usage of oxygen concentrators as a primary oxygen supply over a 10 year period, noting that the concentrators were found to be safe, reliable and cost effective. The US military has used 93% oxygen for many years and declared it as acceptable in any clinical application.

The oxygen produced for use in oxygen cylinders or liquid storage devices is generally 99% pure oxygen produced under a process called Vacuum Swing Adsorption (VSA). The device of the present disclosure will produce oxygen at a purity rate of 93% using the Pressure Swing Adsorption (PSA) process technology as outlined elsewhere in the document.

This raises the question of whether 93% oxygen is as good a quality for patient care as 99% oxygen. According to clinical studies carried out in Great Britain, Canada and the United States clinical care remains the same whether the oxygen supply is 93% or 99%. One study examined the efficacy of different oxygen delivery systems using both 93% and 99% oxygen at 2 L/min, 3 L/min and 4 L/min. Results showed that the inspired oxygen (FiO₂) changed due to the different flow rates. However, at each flow rate there was statistically no difference in the FiO₂ between the different concentrations of oxygen.

The device of the present disclosure complies with International Organization for Standardization (ISO), which has issued identical regulations regarding 93% and 99% oxygen delivery systems. Both the Canadian Standards Association (CSA) and the US military make no distinction between the systems.

The device of the present disclosure uses adsorbent beds containing exclusively zeolite molecular sieves. Several varieties (5AMG, MG3, 13X, and OXYSIV-5) are commercially available, however, most oxygen concentrator manufacturers presently use either Oxysiv 5, Oxysiv 7, KEG415, Oxysiv LiLSX, MS S 624, MS C 544, and AgLiLSX for Pressure Swing Adsorption.

Adsorption rate within a zeolite is dependent on how fast diffusion occurs within the zeolite pores. The rate of diffusion is determined by rate properties that include an adsorbent particle's intrinsic characteristics like the structure, size, and shape of the macropores. The adsorption rate in a zeolite is approximately related to the inverse of the square of the particle radius and is directly proportional to the macropore diffusivity and porosity.

Zeolites are hydrated aluminosilicates. Their structure consists in a three-dimensional framework of AlO₄ and SiO₄ tetrahedrae coordinated by oxygen atoms. Zeolites are cation exchangers. Zeolites are used in a multitude of applications, including adsorption/desorption of liquids and gases, energy storage, cation exchange, and catalysis.

In zeolites, cations are usually responsible for the selectivity to nitrogen. These zeolites adsorb preferentially nitrogen instead of oxygen (usually at a rate of about 4:1) mainly due to the interactions between the cations of the zeolite and the quadrupolar moment of the adsorbed gas. Nitrogen quadrupolar moment is about four times the one of oxygen. Since these cations influence in such a significant way the zeolites adsorption capacity, numerous tries have been conducted with the intent of optimizing the zeolites properties by increasing the number of sites destined for cations, by creating zeolites with a higher content of aluminum; or by the synthesis of zeolites with different combinations of cations.

It has been suggested that the addition of a small amount of silver to the zeolites of the type LiX (giving birth to the zeolites of the type AgLiX) to improve the performance of the adsorbent in oxygen separation from air. The AgLiLSX type zeolite, obtained by exchanging silver ions in LiLSX type zeolites, present high nitrogen adsorption capacities and high selectivity of nitrogen over oxygen, at sub-atmospheric pressures. AgLiLSX type zeolites, such as the 40% silver exchanged zeolite may even present a selectivity of argon over oxygen, which allows the production of high purity oxygen (above 99%). This adsorbent can then be used for high purity oxygen production for medical applications (above 99.5% of oxygen), directly from air, allowing, this way, the production of PSA units for use in campaign hospitals or other places where the circumstances demand the immediate use of large quantities of this type of oxygen or where liquid oxygen cylinders are not enough or even a possibility for fulfilling the needs.

| Adsorbents | Uses |
|---|---|
| Activated | Activated alumina to remove and filter out water vapor |
| Alumina | and impurities in the ambient air, such as carbon |
| | dioxide (Air at 100% humidity is approximately 3% |
| | water vapor). Water can impair and damage the |
| | adsorbents in the PSA adsorption columns. Activated |
| | alumina is necessary to remove water vapor from the |
| | air. |
| Activated | The filter is used to remove smells and odors from the |
| Carbon Filter | exterior air for environments with a high level of |
| | pollutants in the air. |
| Zeolites | Are microporous crystalline structures with a lifespan |
| | of 10 years. The zeolite's structure governs which |
| | molecules are adsorbed. There are various ways of |
| | controlling adsorption. Molecules can be separated |
| | based on differences of size, shape, and polarity. |

### Zeolite Compositions and Properties

| Zeolites | Properties |
|---|---|
| LiLSX | Oxysiv LiLSX is a low silica X-type zeolite |
| Zeolites | (LiLSX) Si/Al = 1. |
| LiAgX | Useful for removing Nitrogen from Oxygen with |
| Zeolite | product throughput .1 kg O₂/hr/kg adsorbent. |
| | The zeolite can obtain 96.42% oxygen purity |
| | with 62.74% Oxygen recovery. Drawback is the |
| | selectivity of Argon to Oxygen is approximately |
| | 1:1. |
| AgX | Argon to Oxygen selectivity of 1.63 to 1, 7 cm³/g |
| Zeolite | of Argon adsorbed at atmospheric pressure and |
| | Nitrogen to Oxygen selectivity of 5 to 1. This |
| | zeolite has greater affinity to Argon over |
| | Oxygen, if you wish to obtain 99% purity with |
| | air, a combination of both LiAgX and AgX is |
| | recommended. |
| 13X and | 13X also known as NaX and 5A also known as |
| 5A Zeolite | CaA. Is a generic sorbent, it has previously |
| | used prior to the discovery of AgX and LiAgX, |
| | separation is done by pore size. While these |
| | zeolites are capable for the separation of O₂ and |
| | N₂ from the air, they have a lower loading |
| | capacity of O₂ and N₂, meaning the portable |
| | device would require more zeolite to perform |
| | at the rate than that of a device which had |
| | LiAgX/LiAgLSX or AgX. |
| Activated | In a preferred embodiment, an activated alumina |
| Alumina | composition is used. The activated alumina |
| | composition may comprise at least one of Al₂O₃, |
| | Na₂O, Fe₂O₃, TiO₂, or SiO₂. |
| LiLSX | In another preferred embodiment, a LiLSX zeolite |
| Zeolite | composition is used. The LiLSX zeolite composition |
| | may comprise at least one of Zeolite, cuboidal, |
| | crystalline, synthetic, non-fibrous, mineral binder, |
| | or Quartz (SiO₂). |

The device of the present disclosure utilizes a unique zeolite recipe. The unique zeolite recipe may comprise activated alumina and LiLSX compositions. As noted above, the activated alumina composition may comprise at least one of Al₂O₃, Na₂O , Fe₂O 3, TiO₂, or SiO₂. The LiLSX composition may comprise at least one of Zeolite, cuboida!, crystalline, synthetic, non-fibrous, mineral binder, or Quartz (SiO₂). Ideally, the smaller the zeolite particles, the better performance allowing for the area of contact of ambient air to increase, allowing higher adsorption to happen. In doing so, allows for better performance in oxygen productivity out the device therefore allowing us to decrease the bed size smaller, in turn decreasing overall POC physical volumetric.

In some embodiments, the zeolites may be about 0.2 mm to about 1.0 mm in diameter. In a preferred embodiment, the zeolites may be about 0.4 mm in diameter.

However the zeolite separation performance cannot be improved indefinitely by reducing the particle size. When particle is too small (<0.4 mm), the mass transfer properties of gas flowthrough the bed changes. The pressure drop across the bed increases, and oxygen recovery rate drops due to increase in quantity of oxygen required to back purge.

According to an embodiment of the present disclosure, the zeolite composition may comprise an activated alumina composition and an LiLSX composition. The weight ratio of the activated alumina composition to the LiLSX composition may be in a range of about 0.2 to about 0.5. In some aspects, the LiLSX composition may comprise a plurality of first pellets. The first pellets may each have a size of about 0.4 mm and a mesh size of about 30×60. In other aspects, the activated alumina composition may comprise a plurality of second pellets. The second pellets may each have a size of about 0.5 mm and a mesh size of about 28×48.

In some embodiments, the pellet size and the mesh size used may match both the LiLSX composition and the activated alumina composition in order to allow the zeolites to undergo same or similar adsorption rate at the same or similar pressure and flow. This may provide optimal results in producing concentrated oxygen to the user.

Our main focus is to develop an optimal conditional setting for the LiLSX material. Its particle size allows us to work within the parameters to reduce overall POC physical volumetric.

In order to gauge our design in terms of performance to current POCs in the market, we took measurements on output oxygen concentration and Continuous Oxygen Production Flow (Liters per minute). Generally, POCs in the market produced in the range of 85-92% oxygen concentration, with the continuous output flow varying from 0.33-3 LPM. The pattern we noticed is that continuous output flow is proportional to the size of POC. The smallest POC weighs at 1.5 kg produces 0.33 LPM continuous, whereas 3 LPM devices weighs up to 9 kg.

We found that most market POCs advertised their products to be able to produce pulse dosage of oxygen in the excess of 6 LPM maximum. It is important to note that pulse dosage only provides oxygen doses when patient is breathing, in order to simulate a continuous flow. This specification cannot be used to measure a true performance of a POC as different suppliers have different assumptions on the average users' breaths per minute and the volume per pulse dose of oxygen.

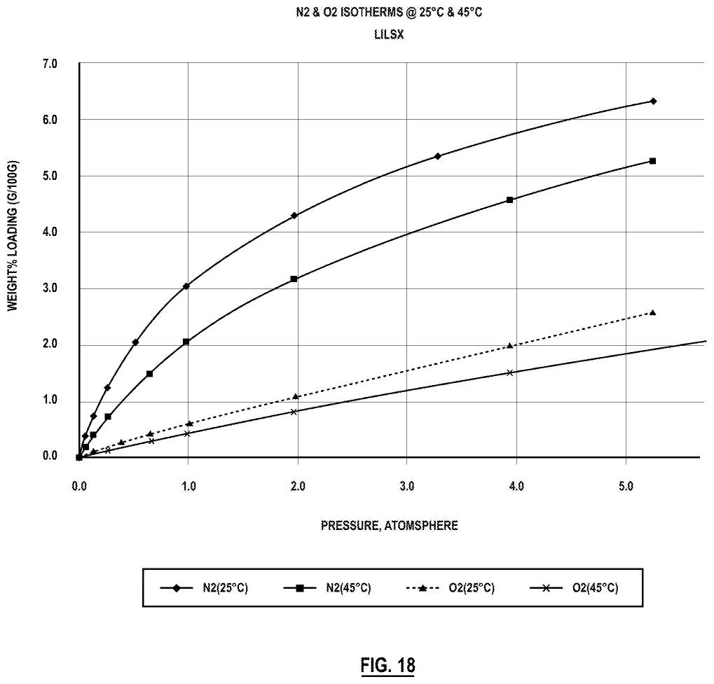

FIG. 18 graphically compares the weight % loading of zeolites in the zeolite bed and the pressure drop measured across the zeolite bed. The device of the present disclosure may operate at a range between about 1.4 Bar (20 psi) to about 2 Bar (29 psi). At 1.4 Bar, for example, the device may be able to produce 85% oxygen purity whereas at 2 Bar, the device may be capable of producing 91% oxygen purity.

While the present disclosure is described herein with reference to illustrative embodiments of manifolds, columns, wave springs, zeolites, etc. used for particular applications, such as for providing concentrated oxygen to a user, it should be understood that the embodiments described herein are not limited thereto. Those having ordinary skill in the art and access to the teachings provided herein will recognize additional modifications, applications, embodiments, and substitution of equivalents that all fall within the scope of the disclosed embodiments. Accordingly, the disclosed embodiments are not to be considered as limited by the foregoing or following descriptions.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described and accordingly, all suitable modifications and equivalents may be resorted to, failing within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description.

1. A portable oxygen concentrator, comprising:
an input configured to receive air flow;
an input filter;
a compressor configured to compress air flow;
a first column comprising a first adsorbent bed;
a second column adjacent to the first column, the second column comprising a second adsorbent bed;
a first output configured to release oxygen to a user; and
a second output configured to release waste gas,
wherein the first and second adsorbent beds comprise a plurality of zeolites.

2. The portable oxygen concentrator of claim 1, further comprising:
a top manifold at a distal end of the first and second columns; and
a bottom manifold at a proximal end of the first and second columns,
wherein the top and bottom manifolds comprise the first and second outputs and an internal network of tubes configured to allow air flow.

3. The portable oxygen concentrator of claim 2, wherein the top and bottom manifolds further comprise a plurality of solenoid valves configured to control the flow of air.

4. (canceled)

5. The portable oxygen concentrator of claim 2, wherein the top and bottom manifolds further comprise a plurality of holes configured to control a flow rate of air.

6. (canceled)

7. The portable oxygen concentrator of claim 5, further comprising a plurality of check valves on the top and bottom manifolds configured to seal the plurality of holes.

8.-10. (canceled)

11. The portable oxygen concentrator of claim 1, wherein:
a proximal end of the first and second columns is coupled to the first output;
a distal end of the first and second columns is coupled to the compressor; and
the first and second columns each comprise an O-ring coupled to at least one of the proximal or distal end.

12. The portable oxygen concentrator of claim 11, further comprising a cap at the proximal end and the distal end of the first and second columns, wherein the cap comprises a tapered air flow path.

13. The portable oxygen concentrator of claim 12, further comprising at least one sintered glass filter disc at the proximal end and the distal end of the first and second columns, wherein the at least one sintered glass filter disc is configured to filter the plurality of zeolites from the compressed air.

14. The portable oxygen concentrator of claim 13, further comprising a wave spring located in between the cap and the at least one sintered glass filter disc, wherein the wave spring is configured to compress the plurality of zeolites in the first and second columns.

15. The portable oxygen concentrator of claim 13, further comprising dense foam material located in the cap, wherein the dense foam material is configured to compress the plurality of zeolites in the first and second columns.

16. The portable oxygen concentrator of claim 13, further comprising a rubber durometer located in the cap, wherein the rubber durometer is configured to compress the plurality of zeolites in the first and second columns.

17. (canceled)

18. The portable oxygen concentrator of claim 1, further comprising at least one sensor and a processor, wherein:
the at least one sensor is configured to detect at least one physiological parameter of the user; and
the processor is configured to adjust an amount of oxygen released to the user based on the detected at least one physiological parameter.

19. The portable oxygen concentrator of claim 18, wherein the at least one sensor comprises at least one of a pulse oximeter, differential pressure sensor, ECG, EEG, gyroscope, or accelerometer.

20. (canceled)

21. (canceled)

22. The portable oxygen concentrator of claim 18, wherein the processor is further configured to generate an alarm when the detected at least one physiological parameter is above or below a predetermined threshold.

23.-26. (canceled)

27. The portable oxygen concentrator of claim 1, wherein:
the first column is configured to provide oxygen to the first output when the second column is configured to release waste gas to the second output; and
the first column is configured to release waste gas to the second output when the second column is configured to provide oxygen to the first output.

28. (canceled)

29. The portable oxygen concentrator of claim 1, wherein the first and second columns are configured to allow air to flow radially to thereby channel the air through the first and second columns and increase contact with the plurality of zeolites in the first and second adsorbent beds.

30. The portable oxygen concentrator of claim 1, wherein the first and second columns each comprise between about 20 and about 80 grams of zeolites.

31. (canceled)

32. (canceled)

33. The portable oxygen concentrator of claim 18, further comprising a user interface configured to receive user input, wherein the processor is configured to adjust the amount of oxygen released to the user based on the user input.

34. The portable oxygen concentrator of claim 18, further comprising a wireless receiver configured to receive data from a remote device, wherein the processor is configured to adjust the amount of oxygen released to the user based on the received data.

35. (canceled)

36. (canceled)

37. A method of providing concentrated oxygen to a user, the method comprising:
directing and compressing air into a first column of an oxygen concentrator, wherein the first column comprises a first adsorbent bed;
absorbing nitrogen and oxygen molecules from the air in the first adsorbent bed;
directing and compressing the air into a second column of an oxygen concentrator adjacent to the first column, wherein the second column comprises a second adsorbent bed;
absorbing the nitrogen and oxygen molecules from the air in the second adsorbent bed;
depressurizing the first column, wherein depressurizing the first column allows the argon and nitrogen molecules in the first column to be purged out of the oxygen concentrator and released to the atmosphere;
directing and compressing the air into the first column; and
depressurizing the second column, wherein depressurizing the second column allows the argon and nitrogen molecules in the second column to be purged out of the oxygen concentrator and released to the atmosphere.

38. The method of claim 37, wherein depressurizing the first column and directing and compressing the air into the second column are performed concurrently.

39. (canceled)
